(19) United States
(12) Patent Application Publication
(10) Pub. No.: US 2021/0277457 A1
(43) Pub. Date: Sep. 9, 2021

(54) METHOD FOR ACCURATE QUANTIFICATION OF GENOMIC COPIES IN CELL-FREE DNA

(71) Applicant: GRAIL, Inc., Menlo Park, CA (US)

(72) Inventors: Hamed AMINI, Menlo Park, CA (US); Sudha NAGARAJU, Menlo Park, CA (US); Alex ARAVANIS, Menlo Park, CA (US); Arash JAMSHIDI, Menlo Park, CA (US)

(21) Appl. No.: 16/325,122

(22) PCT Filed: Aug. 11, 2017
(86) PCT No.: PCT/US2017/046582

§ 371 Date: Feb. 12, 2019

(51) Int. Cl. C12Q 1/6851; C12Q 1/686; C12Q 1/6855; C12Q 1/6869

(52) U.S. Cl. CPC C12Q 1/6851; C12Q 1/686; C12Q 2563/185; C12Q 1/6869; C12Q 1/6855

(57) ABSTRACT

Described herein are methods and systems for quantifying low molecular weight nucleic acid molecules in a biological sample amongst a background of high molecular weight contamination.

### CROSS REFERENCE TO RELATED APPLICATIONS

The present application claims benefit of priority of U.S. Provisional Application Ser. No. 62/374,674 filed on Aug. 12, 2016; and U.S. Provisional Application Ser. No. 62/394,139 filed on Sep. 13, 2016; both of which are herein incorporated by reference in their entirety.

### BACKGROUND

There are a number of methods that are currently used to determine genome copy number in a cell-free DNA (cfDNA) sample. Indirect or mass measurement methods (e.g., Fragment Analyzer™, BioAnalyzer, qPCR, plate reader) require normalization to a control (e.g., either to a reference or to a standard curve), allowing only a relative determination of the amount (mass) of nucleic acid in a cfDNA sample. The measured mass is then converted to the number of haploid genome copies (i.e., about 3 pg per haploid genome). Direct counting methods (e.g., droplet-based digital PCR (ddPCR)) that target a reference gene (“single-point” ddPCR) in a sample provide absolute counting of target copies without the need for a standard curve. However, in the context of a cfDNA sample, wherein the average fragment size is relatively small (average size about 160-170 bp), single-point ddPCR quantification may be inaccurate leading to under-counting of target copies (i.e., short cfDNA fragments may not be amplified due to lack of primer binding). Further, contamination by higher molecular weight gDNA in the cfDNA sample can lead to over-counting of target copies (i.e., primers and probes targeted to cfDNA amplify higher molecular weight gDNA if present). There is a need for new methods for accurate genome copy number quantification of cfDNA in a sample.

### SUMMARY

The methods and systems described herein are useful for improving the quantitation of low molecular weight nucleic acids (e.g., cell-free nucleic acids) amongst a background of high molecular weight nucleic acids (e.g., cell associated RNAs and genomic DNA) in a sample. The sample can be a biological sample obtained by a minimally invasive collection method, such as, for example a blood draw, stool sample, saliva sample, or urine sample. The cell-free nucleic acids are nucleic acids that exist outside of a cell before the sample is obtained, and the high molecular weight nucleic acids are nucleic acids that exist inside the cell at the time the sample is obtained. High molecular weight nucleic acids can also come from exogenous contamination during sample prep and analysis (e.g., cross-contamination). The low molecular weight targets selected may be selected due to their utility in the diagnosis and/or by monitoring of a disease such as a cancer/tumor, transplant status or fetal status. In some embodiments, the method comprises quantifying both low molecular weight targets and high molecular weight targets (e.g., total nucleic acid targets) in one subsample of a biological sample, comparing this quantitation to the high molecular weight nucleic acid targets in another subsample, and correcting for the amount of high molecular weight contamination.

Described herein in one aspect is a method for quantifying low molecular weight nucleic acid molecules in a biological sample comprising said low molecular weight nucleic acid molecules and high molecular weight nucleic acid molecules, comprising: (a) on a first subsample of said biological sample, quantifying total nucleic acid targets, wherein said total nucleic acids comprise both low molecular weight nucleic acid targets and high molecular weight nucleic acid targets; (b) on a second subsample of said biological sample, quantifying one or more high molecular weight nucleic acid targets, wherein said high molecular weight nucleic acid targets are longer than said low molecular weight nucleic acid targets; and (c) quantifying said low molecular weight nucleic acid targets in said biological sample by comparing an amount of said high molecular weight nucleic acid targets and said low molecular weight nucleic acid targets. In certain embodiments, said high molecular weight nucleic acid targets, said low molecular weight nucleic acid targets, or both comprise DNA molecules. In certain embodiments, digital PCR (dPCR) is used to quantify one or more of said high molecular weight nucleic acid targets, one or more of said low molecular weight nucleic acid targets, or both of said high molecular weight nucleic acid targets and said low molecular weight nucleic acid targets. In certain embodiments, said low molecular weight nucleic acid targets are shorter than about 700 base pairs. In certain embodiments, said low molecular weight nucleic acid targets are between about 150 to about 190 base pairs. In certain embodiments, said high molecular weight nucleic acid targets are longer than about 700 base pairs. In certain embodiments, said high molecular weight nucleic acid targets are between about 700 to about 2000 base pairs. In certain embodiments, said low molecular weight DNA targets comprise cell-free DNA (cfDNA) present when said biological sample was obtained from an individual. In certain embodiments, said high molecular weight DNA targets comprise genomic DNA inside a cell when said biological sample was obtained from an individual. In certain embodiments, said low molecular weight nucleic acid targets are highly conserved regions of the genome. In certain embodiments, said high molecular weight nucleic acid targets are highly conserved regions of the genome. In certain embodiments, the average length of said low molecular weight nucleic acid targets are is less than about 300 base pairs. In certain embodiments, the average length of said low molecular weight nucleic acid targets is less than about 170 base pairs. In certain embodiments, the average length of said low molecular weight nucleic acid targets range from about 60 to about 100 base pairs. In certain embodiments, the average length of said high molecular weight nucleic acid targets is greater than about 300 base pairs. In certain embodiments, the average length of said high molecular weight nucleic acid targets range from about 300 to about 600 base pairs. In certain embodiments, the average length of said high molecular weight nucleic acid targets is greater than about 700 base pairs. In certain embodiments, said low molecular weight nucleic acid targets comprise a plurality of low molecular weight nucleic acid targets selected to yield amplicons of different lengths across a genome. In certain embodiments, said high molecular weight nucleic acid targets comprise a plurality of high molecular weight nucleic acid targets selected to yield amplicons of different lengths across a genome. In certain embodiments, said low molecular weight nucleic acid targets, said high molecular weight nucleic acid targets, or both said low molecular weight nucleic acid targets and said high molecular weight nucleic acid targets are quantified by a plurality of primer pairs that selectively hybridize to highly conserved regions of the genome. In certain embodiments, said plurality of primer pairs used to quantify said low molecular weight nucleic acid targets selected to yield at least two or more different length amplicons across at least two or more different target regions of the genome. In certain embodiments, said plurality of primer pairs used to quantify said low molecular weight nucleic acid targets selected to yield at least 7 different length amplicons across at least 4 different target regions of the genome. In certain embodiments, said low molecular weight nucleic acid targets, said high molecular weight nucleic acid targets, or both said low molecular weight nucleic acid targets and said high molecular weight nucleic acid targets are quantified by a plurality of primer pairs that selectively hybridize to highly conserved regions of the genome. In certain embodiments, the plurality of primer pairs to quantify the low molecular weight nucleic acid targets are selected to yield at least two or more different length amplicons across at least two or more different target regions of the genome. In certain embodiments, the biological sample is selected from the list consisting of whole-blood, plasma, serum, saliva, lymph, and urine.

Also described herein, is a computer-implemented system comprising: a computer comprising: at least one processor, a memory, an operating system configured to perform executable instructions, and a computer program including instructions executable by the at least one processor to create an application that quantifies low molecular weight nucleic acid molecules, the application that quantifies low molecular weight nucleic acid molecules configured to perform the following: (a) quantify total nucleic acid targets from a reaction performed on a subsample, wherein said total nucleic acids comprise both low molecular weight nucleic acid targets and high molecular weight nucleic acid targets; (b) quantify one or more high molecular weight nucleic acid targets from a reaction performed on a subsample, wherein said high molecular weight nucleic acid targets are longer than said low molecular weight nucleic acid targets; and (c) quantify said low molecular weight nucleic acid targets in said biological sample by comparing an amount of said high molecular weight nucleic acid targets and said low molecular weight nucleic acid targets. In certain embodiments, said high molecular weight nucleic acid targets, said low molecular weight nucleic acid targets, or both comprise DNA molecules. In certain embodiments, digital PCR (dPCR) is used to quantify one or more of said high molecular weight nucleic acid targets, one or more of said low molecular weight nucleic acid targets, or both of said high molecular weight nucleic acid targets and said low molecular weight nucleic acid targets. In certain embodiments, said low molecular weight nucleic acid targets are shorter than about 700 base pairs. In certain embodiments, said low molecular weight nucleic acid targets are between about 150 to about 190 base pairs. In certain embodiments, said high molecular weight nucleic acid targets are longer than about 700 base pairs. In certain embodiments, said high molecular weight nucleic acid targets are between about 700 to about 2000 base pairs. In certain embodiments, said low molecular weight DNA targets comprise cell-free DNA (cfDNA) present when said biological sample was obtained from an individual. In certain embodiments, said high molecular weight DNA targets comprise genomic DNA inside a cell when said biological sample was obtained from an individual. In certain embodiments, said high molecular weight nucleic acid targets are highly conserved regions of the genome. In certain embodiments, said high molecular weight nucleic acid targets are highly conserved regions of the genome. In certain embodiments, the average length of said low molecular weight nucleic acid targets are is less than about 300 base pairs. In certain embodiments, the average length of said low molecular weight nucleic acid targets is less than about 170 base pairs. In certain embodiments, the average length of said low molecular weight nucleic acid targets range from about 60 to about 100 base pairs. In certain embodiments, the average length of said high molecular weight nucleic acid targets is greater than about 300 base pairs. In certain embodiments, the average length of said high molecular weight nucleic acid targets range from about 300 to about 600 base pairs. In certain embodiments, the average length of said high molecular weight nucleic acid targets is greater than about 700 base pairs. In certain embodiments, said low molecular weight nucleic acid targets comprise a plurality of low molecular weight nucleic acid targets selected to yield amplicons of different lengths across a genome. In certain embodiments, said high molecular weight nucleic acid targets comprise a plurality of high molecular weight nucleic acid targets selected to yield amplicons of different lengths across a genome. In certain embodiments, said low molecular weight nucleic acid targets, said high molecular weight nucleic acid targets, or both said low molecular weight nucleic acid targets and said high molecular weight nucleic acid targets are quantified by a plurality of primer pairs that selectively hybridize to highly conserved regions of the genome. In certain embodiments, said plurality of primer pairs used to quantify said low molecular weight nucleic acid targets selected to yield at least two or more different length amplicons across at least two or more different target regions of the genome. In certain embodiments, said plurality of primer pairs used to quantify said low molecular weight nucleic acid targets selected to yield at least 7 different length amplicons across at least 4 different target regions of the genome. In certain embodiments, said low molecular weight nucleic acid targets, said high molecular weight nucleic acid targets, or both said low molecular weight nucleic acid targets and said high molecular weight nucleic acid targets are quantified by a plurality of primer pairs that selectively hybridize to highly conserved regions of the genome. In certain embodiments, the plurality of primer pairs to quantify the low molecular weight nucleic acid targets are selected to yield at least two or more different length amplicons across at least two or more different target regions of the genome. In certain embodiments, the biological sample is selected from the list consisting of whole-blood, plasma, serum, saliva, lymph, and urine.

In another aspect described herein is a method for determining a conversion efficiency in one or more steps of a nucleic acid sequencing and analysis workflow, the method comprising: (a) performing a step of said nucleic acid sequencing and analysis workflow on a sample comprising low molecular weight nucleic acid targets and high molecular weight nucleic acid targets; and (b) quantifying, using a digital PCR (dPCR) amplification reaction, a number of said low molecular weight nucleic acid targets in said sample before and after said step of the sequencing and analysis workflow, and comparing the number of low molecular weight nucleic acid targets in the sample before and after said sequencing and analysis workflow to determine said conversion efficiency of the step. In certain embodiments, said dPCR amplification reaction comprises droplet digital polymerase chain (ddPCR). In certain embodiments, said one or more steps of said sequencing and analysis workflow is selected from the group consisting of: DNA isolation, enrichment, ligating adaptors, performing a universal amplification step, attaching barcodes, and sequencing. In certain embodiments, said step of said sequencing and analysis workflow is a plurality of steps selected from the group consisting of: DNA isolation, enrichment, ligating adaptors, performing a universal amplification step, attaching barcodes, and sequencing. In certain embodiments, said low molecular weight nucleic acid targets are quantified by a plurality of primer pairs that selectively hybridize to highly conserved regions of the genome. The method of claim 26, wherein quantifying comprises determining a first target count using a first set of one or more primer pairs that amplify one or more first regions of the genome and a second target count using a second set of one or more primer pairs that amplify one or more second regions of the genome. In certain embodiments, estimating the conversion efficiency comprises comparing the second target count and the first target count. In certain embodiments, the step is repeated if said conversion efficiency is less than about 20%. In certain embodiments, the average length of said low molecular weight nucleic acid targets are is less than about 300 base pairs. In certain embodiments, the average length of said low molecular weight nucleic acid targets is less than about 170 base pairs. In certain embodiments, the average length of said low molecular weight nucleic acid targets range from about 60 to about 100 base pairs.

### BRIEF DESCRIPTION OF THE DRAWINGS

FIG. 1 illustrates a flow diagram of an example of a method for accurate copy number quantification in cfDNA;

FIG. 2 shows a schematic plot of ddPCR counts as a function of amplicon length (lₐ);

FIG. 3A shows a plot of droplet fluorescence from an experiment in which high molecular weight gDNA fragments are selectively amplified in a cfDNA sample;

FIG. 3B shows a representative plot of the fragment size distribution of the sample used in the plot shown in FIG. 3A;

FIG. 4 shows a plot of ddPCR counts corrected for large gDNA contamination;

FIG. 5 shows a plot of the fragment size distribution of size-selected genomic DNA used to evaluate counts (N_{c}) as a function of amplicon length;

FIGS. 6A and 6B show a plot of counts (N_{c}) as a function of amplicon length in a un-sheared high molecular weight gDNA sample and a plot of counts (N_{c}) as a function of amplicon length in the size-selected sheared gDNA of FIG. 5, respectively;

FIG. 7A shows a density plot of fragment size distribution for a single size fragment;

FIG. 7B shows a plot of the frequency of fragment density as a function of fragment size (bp) for a hypothetical sample with consisting of various fragment sizes;

FIG. 7C shows a plot of a typical cfDNA sample fragment size distribution;

FIGS. 8A and 8B show a plot of function ln(x) and a plot of function x.ln(x) and their linear behavior for the range of 60-100 (corresponding to amplicon lengths), respectively;

FIGS. 9A and 9B show a plot of the simulation of fragment density as a function of fragment length and a plot of the simulation of the expected output efficiency as function of amplicon length, respectively;

FIGS. 10A, 10B, 10C, and 10D show plots of counts (N_{c}) as a function of amplicon length for 4 different cfDNA samples, NS-02, NS-03, NS-11, and NS-17, respectively;

FIG. 11 illustrates a flow diagram of an example of a method of estimating the conversion efficiency in a cfDNA sequencing and analysis workflow;

FIG. 12 shows a bar graph of a comparison of cfDNA quantification using ddPCR copy number quantification and Fragment Analyzer™ quantification; and

FIG. 13 illustrates a flow diagram of an example of a method of using conversion efficiency in a cfDNA workflow to provide a level of confidence for a diagnostic test result.

FIG. 14 shows a non-limiting example of a digital processing device; in this case, a device with one or more CPUs, a memory, a communication interface, and a display.

### DETAILED DESCRIPTION

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein the term “about” refers to an amount that is near the stated amount by 10%, 5%, or 1%.

Described herein is a method for accurate haploid genome copy number quantification of cfDNA in a sample. In some embodiments, the method uses digital PCR (e.g., droplet-based digital PCR (ddPCR)) to count target DNA molecules in a cfDNA sample, wherein a first ddPCR assay is used to amplify and count a set of unique target DNA molecules (e.g., cfDNA and gDNA amplicons) and a second ddPCR assay is used to selectively amplify and count high molecular weight gDNA molecules (gDNA amplicons) in the cfDNA sample.

In one embodiment, the first ddPCR assay is performed using a set of amplification primer pairs and probes selected to yield amplicons of different lengths (e.g., ranging from about 60 to about 100 bp) targeted across highly conserved regions of the genome. The measurement (count) of target DNA molecules is then used to impute (estimate) the number of haploid genome copies in the original cfDNA sample. The second ddPCR assay is performed using a single amplification primer pair and probe selected to yield a relatively long amplicon (e.g., about 300-600 bp) targeted to a certain highly conserved region of the genome. The second ddPCR assay is used to distinguish cfDNA (e.g., cfDNA<about 700 bp) from higher molecular weight gDNA (e.g., gDNA>about 700 bp). The count of target gDNA molecules is used to adjust the count of target cfDNA molecules (obtained in the first ddPCR assay), correcting for high molecular weight gDNA contamination.

In one application, the method is used to provide a measurement of unique molecule input (haploid genome equivalents (hGE)) for estimation of the conversion efficiency in a cfDNA workflow, e.g., a cfDNA sequencing and analysis workflow. Conversion efficiency (ii) can be described as workflow output (e.g., number of unique molecules read after sequence analysis) divided by sample input (e.g., number of unique molecules input). The conversion efficiency can be determined for one or more steps in a cfDNA workflow. In one example, the conversion efficiency for one or more steps in a cfDNA workflow can be used in an assay development and/or improvement process. This approach can be extended to quantify number of unique molecules converted at different steps of the cfDNA workflow (e.g., after library prep) to determine efficiency at different stages.

In another application, the method is used for quality control (QC) in a molecular diagnostic test (e.g., a next generation sequencing (NGS) diagnostic test), wherein the QC step is used to determine the conversion efficiency (i.e., workflow output divided by sample input) in a cfDNA workflow and provide a level of confidence for the diagnostic test result.

In yet another application, the method can be used as a discovery tool to differentiate and count different components in a cfDNA sample (e.g., ssDNA, damaged DNA, etc.).

In some embodiments, the methods described herein are used for accurately quantitating low molecular weight nucleic acids in a biological sample. In certain embodiments, the biological sample is acquired using minimally invasive techniques. In certain embodiments, the biological sample comprises whole blood, serum, plasma, urine, fecal matter, saliva, semen, vaginal fluid, or a core biopsy sample. In certain embodiments, the biological sample comprises whole blood, serum, or plasma. The low molecular weight nucleic acids quantitated can be DNA, RNA, siRNA, or single stranded DNA molecules. The methods described herein also accurately quantitate high molecular weight nucleic acid contamination in a biological sample. In certain embodiments, the biological sample is acquired using minimally invasive techniques. In certain embodiments, the biological sample comprises whole blood, serum, plasma, urine, fecal matter, saliva, semen, vaginal fluid, or a core biopsy sample. In certain embodiments, the biological sample comprises whole blood, serum, or plasma. The low molecular weight nucleic acids quantitated can be DNA, RNA, siRNA or single stranded DNA molecules.

Accurately quantitating low molecular weight nucleic acids is useful in the diagnosis of cancer, monitoring of response to cancer treatment, monitoring organ transplant status, or monitoring fetal status. In certain embodiments, the methods described herein are for use in monitoring cancer treatment.

The low molecular weight nucleic acid targets of the present disclosure comprise cfDNA fragments which are generally short in terms of length. In certain embodiments, the low molecular weight nucleic acid targets are less than about 800, 700, 600, 500, 400, 300, 250, 200, 190, 180, 170, 160, 150, or 100 base pairs. In certain embodiments, the average length of the low molecular weight nucleic acid targets are less than about 800, 700, 600, 500, 400, 300, 250, 200, 190, 180, 170, 160, 150, or 100 base pairs. In certain embodiments, the low molecular weight nucleic acid targets are between about 300 and about 100 base pairs in length, between about 250 and about 150 base pairs in length, between about 225 and about 150 base pairs in length, between about 200 and about 150 base pairs in length, between about 190 and about 150 base pairs in length, between about 180 and about 150 base pairs in length, between about 180 and about 160 base pairs in length, between about 180 and about 170 base pairs in length, between about 180 and about 160 base pairs in length, between about 170 and about 160 base pairs in length.

The high molecular weight nucleic acid targets of the present disclosure comprise genomic DNA fragments which are longer than the low molecular weight nucleic acid targets. The high molecular weight nucleic acid targets represent unwanted contamination from cell associated DNA that is released into a biological sample by cell lysis. Cell lysis generally occurs during sample collection, sample freezing, sample transport, or sample preparation. Genomic DNA contamination can be differentiated from cfDNA based on its length. In certain embodiments, the high molecular weight nucleic acid targets are greater than about 200, 300, 400, 500, 600, 700, 800, 900, or 1000 base pairs. In certain embodiments, the average length of the high molecular weight nucleic acid targets are greater than about 200, 300, 400, 500, 600, 700, 800, 900, or 1000 base pairs. In certain embodiments, the high molecular weight nucleic acid targets are between about 200 and about 300 base pairs in length, between about 300 and about 2500 base pairs in length, between about 400 and about 2000 base pairs in length, between about 500 and about 2000 base pairs in length, between about 600 and about 2000 base pairs in length, between about 700 and about 2000 base pairs in length, between about 700 and about 1500 base pairs in length, between about 800 and about 1500 base pairs in length, between about 900 and about 1500 base pairs in length, between about 1000 and about 15000 base pairs in length.

Either the low molecular weight or high molecular weight targets can be amplified by 1 or more primer pairs. In certain embodiments, the low molecular weight or high molecular weight targets can be amplified by 2, 3, 4, 5, 6, 7, 8, 9, or 10 unique primer pairs. The unique primer pairs can be targeted to 1, 2, 3, 4, 5, 6, 7, 8, 9, or 10 different genomic regions. Design of these primers can take into account evolutionary conservation to allow the same primer pairs to be used for a large cross-section of unrelated individuals. In certain embodiments, the primer pairs target highly conserved regions of the genome. In certain embodiments, the primer pairs target genes or regions of the genome that are not involved in a disease such as cancer. In certain embodiments, the primer pairs that target low and high molecular weight nucleic acid targets do not create overlapping products.

Multi-Point ddPCR Copy Number Quantification (CNQ)

In some embodiments, the method uses ddPCR to count multiple target DNA molecules in a cfDNA sample. In one example, ddPCR copy number quantification (ddPCR CNQ) is performed using a Droplet Digital™ PCR System and ddPCR Supermix available from Bio-Rad. In another example, ddPCR copy number quantification is performed using a droplet-based digital PCR system available from RainDance Technologies.

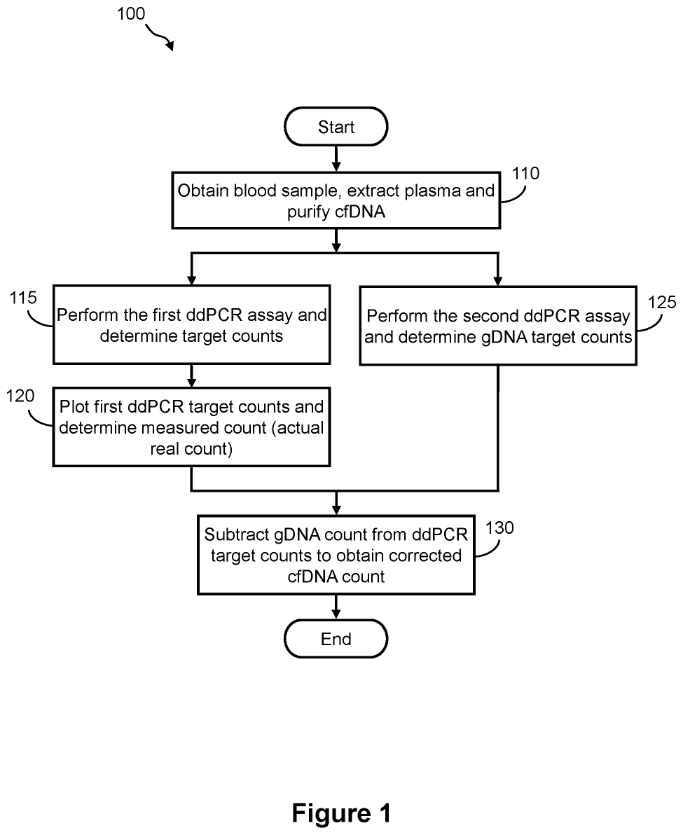

FIG. 1 illustrates a flow diagram of an example of a method 100 for accurate genome copy number quantification of cfDNA in a sample. Method 100 includes, but is not limited to, the following steps.

In a step 110, a blood sample is obtained and cfDNA is isolated from the plasma fraction. In one example, the cfDNA in the plasma fraction is isolated using a QlAamp Circulating Nucleic Acid Kit (available from Qiagen). The sample can then be split for different measurements, e.g., a first ddPCR assay 115 and a second ddPCR assay 125. Method 100 proceeds to both step 115 and 125.

In a step 115, the first ddPCR assay is performed and the absolute number of droplets containing target DNA is determined. For example, the first ddPCR assay is performed (e.g., in duplicate or triplicate) using a set of amplification primer pairs and probes targeted to certain highly conserved regions of the genome. In one example, the set of primer pairs is selected to yield 7 different length amplicons (e.g., ranging from about 60 to about 100 nt) across 4 different target regions of the genome. For each target amplicon, a ddPCR count is determined. A count of 1 target amplicon indicates 1 copy of the genome is present in the cfDNA subsample. In another example, several amplicons of different lengths can be designed on individual regions across the genome, the same type of measurement and calculation performed for each region and then the quantities averaged. Method 100 proceeds to step 120.

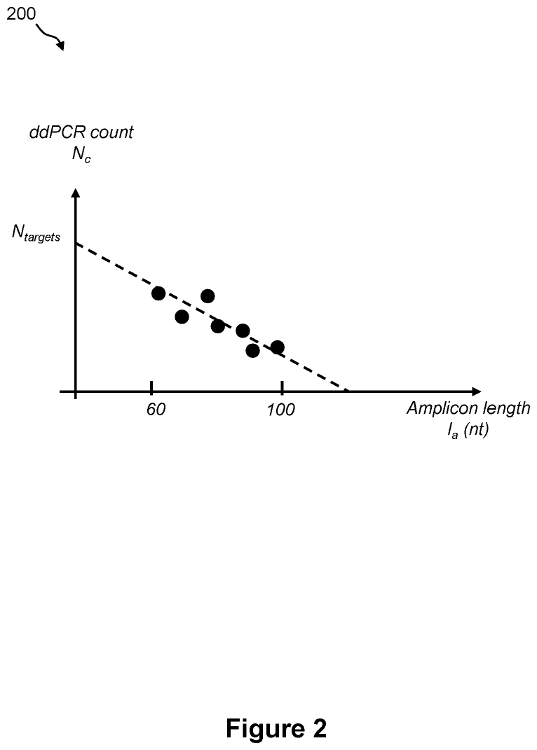

In a step 120, ddPCR counts (N_{c}) for each target from the first ddPCR assay are plotted as a function of amplicon length and a linear regression is fit through the data points to determine the actual real count (or measured count) of target fragments in the cfDNA subsample. Determination of the actual real count (measured count) is described in more detail with reference to FIG. 2. Method 100 proceeds to step 130.

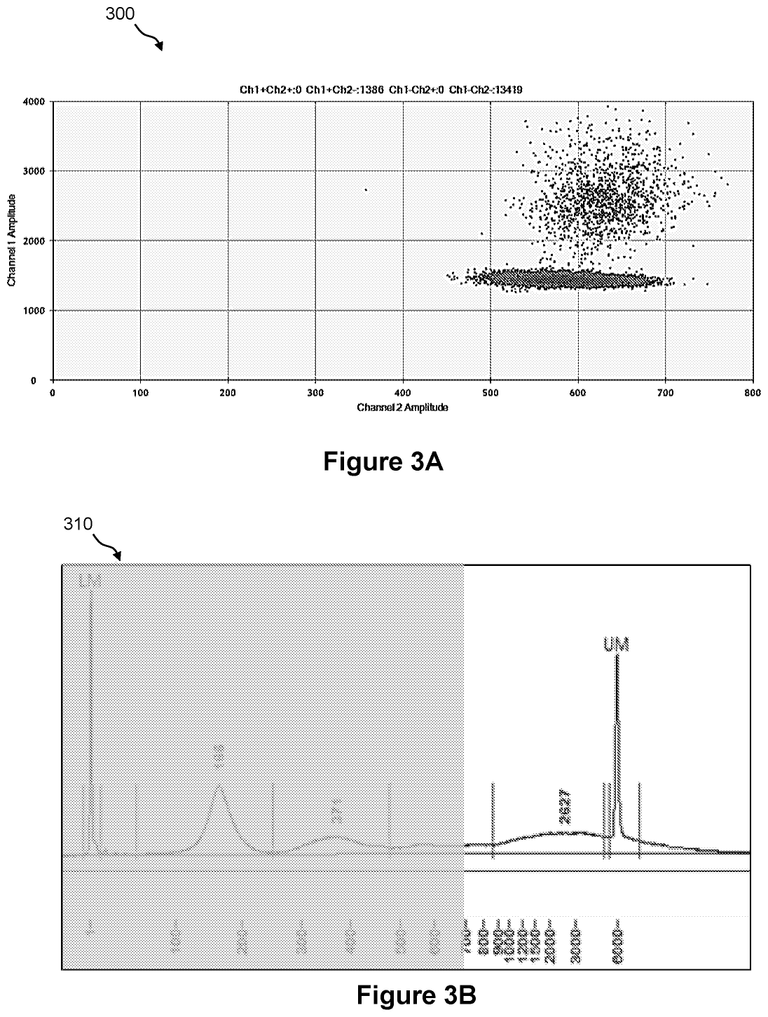

In a step 125, which runs concurrently with steps 115 and 120, the second ddPCR amplification is performed and the absolute number of droplets containing target gDNA is determined. In one example, the second amplification is performed using a single primer pair and probe targeted to a certain highly conserved region of the genome. In another example, the second amplification is performed using two primer pairs and probes targeted to certain highly conserved regions of the genome. The target region(s) of the genome can be, for example, the same region(s) as a region targeted in the first ddPCR amplification. In this amplification reaction, the primer pair(s) is selected to yield a relatively long amplicon (e.g., about 300-600 nt) that is used to count high molecular weight gDNA fragments (e.g., gDNA>about 700 bp) in the cfDNA subsample. Selective amplification of high molecular weight gDNA is described in more detail with reference to FIGS. 3A and 3B. Method 100 proceeds to step 130.

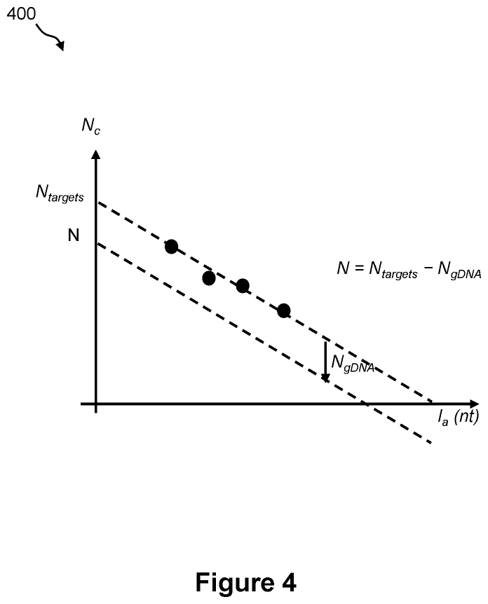

In a step 130, the linear fit count (real count) obtained from the first ddPCR assay is corrected for high molecular weight gDNA contamination, as described in more detail with reference to FIG. 4, which shows a plot for correcting the linear fit count (real count) for large gDNA contamination. To correct for high molecular weight gDNA contamination in a cfDNA subsample, the gDNA count (N_{gDNA}) is subtracted from the linear fit count (N_{targets}) to generate a real corrected count (N_{corr.}) for copy number (i.e., N_{corr.}=N_{targets}−N_{gDNA}). The linear fit line is adjusted downward and the value on the y-axis at that point represents the actual corrected real count of target fragments in the cfDNA subsample. The actual real count of target fragments in the ddPCR subsample is then used to calculate the genome copy number in the original cfDNA sample. While copy number is a primary readout this number can be expresses as an amount per volume, for example, weight by volume (e.g., pg/mL, ng/mL).

In some embodiments, the method is based, in part, on the hypothesis that in a cfDNA sample, the longer a target amplicon is, the lower the number of ddPCR counts will be. For example, for a cfDNA sample where the average fragment size is about 160 bp, if a target amplicon size is 200 bp, the ddPCR counts should be zero because cfDNA fragments in the sample are less than 200 base pairs.

Estimation of Conversion Efficiency Based on ddPCR Copy Number Quantification

In one application, the method is used to provide a measurement of unique molecule input for estimation of the conversion efficiency in a cfDNA workflow. Conversion efficiency (TO can be described as workflow output divided by sample input (i.e., η=output/input). In one example, the total conversion efficiency (ηₜₒₜₐₗ) for a cfDNA sequencing and analysis workflow can be defined as the number of unique molecules read after sequence analysis divided by the number of unique molecules input (i.e., ηₜₒₜₐₗ=number of unique molecules read after analysis/number of unique molecules input).

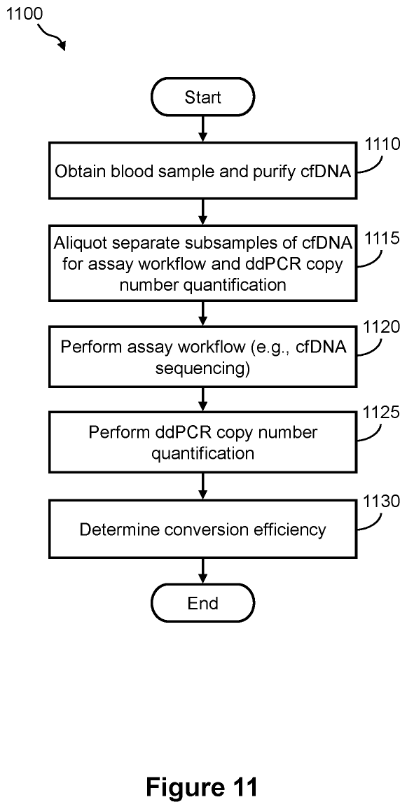

FIG. 11 illustrates a flow diagram of an example of a method 1100 of estimating the conversion efficiency in a cfDNA sequencing and analysis workflow. Method 1100 includes, but is not limited to, the following steps.

In a step 1110, a blood sample is obtained and cfDNA is isolated from the plasma fraction.

In a step 1115, separate subsamples of the cfDNA sample are aliquoted for a cfDNA sequencing and analysis workflow and ddPCR copy number quantification.

In a step 1120, the cfDNA sequencing and analysis workflow is performed. The cfDNA workflow includes, for example, library preparation (e.g., end-repair, A-tailing, ligation, and PCR), library enrichment, sequencing and sequence data analysis.

In a step 1125, ddPCR copy number quantification is performed using method 100 of FIG. 1. ddPCR copy number quantification is used to determine the number of unique molecules input into the cfDNA sequencing and analysis workflow. The calculated copy number per μl (N) for the ddPCR subsample is then used to determine the number of unique molecules input in the cfDNA sequencing and analysis workflow.

In a step 1130, the conversion efficiency is determined. The conversion efficiency (ηₜₒₜₐₗ) for a cfDNA sequencing and analysis workflow is defined as the number of unique molecules read after sequence analysis divided by the number of unique molecules input (i.e., ηₜₒₜₐₗ=mean collapsed coverage/estimated input by ddPCR copy number quantification).

Diagnostic Application of ddPCR Copy Number Quantification

In another application, the method is used for quality control (QC) in a molecular diagnostic test (e.g., a next generation sequencing (NGS) diagnostic test), wherein the QC step is used to determine the conversion efficiency (i.e., workflow output divided by sample input) in a cfDNA workflow and provide a level of confidence for the diagnostic test result.

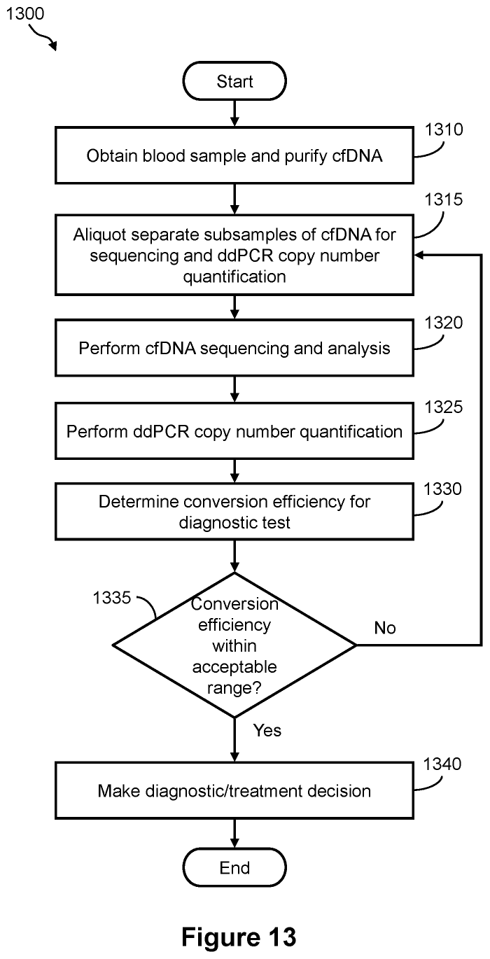

FIG. 13 illustrates a flow diagram of an example of a method 1300 of using conversion efficiency in a cfDNA workflow to provide a level of confidence for a diagnostic test result. In this example, the cfDNA workflow is a cfDNA sequencing and analysis workflow. Method 1300 includes, but is not limited to, the following steps.

In a step 1310, a blood sample is obtained and cfDNA is isolated from the plasma fraction.

In a step 1315, separate subsamples of the cfDNA sample are aliquoted for a cfDNA sequencing and analysis workflow and ddPCR copy number quantification.

In a step 1320, the cfDNA sequencing and analysis workflow is performed. The cfDNA workflow includes, for example, library preparation (e.g., end-repair, A-tailing, ligation, and PCR), library enrichment, sequencing and sequence data analysis.

In a step 1325, ddPCR copy number quantification is performed using method 100 of FIG. 1. ddPCR copy number quantification is used to determine the number of unique molecules input into the cfDNA sequencing and analysis workflow. The calculated copy number per μl (N) for the ddPCR subsample is then used to determine the number of unique molecules input in the cfDNA sequencing and analysis workflow.

In a step 1330, the conversion efficiency is determined. The conversion efficiency (ηₜₒₜₐₗ) for a cfDNA sequencing and analysis workflow is defined as the number of unique molecules read after sequence analysis divided by the number of unique molecules input (i.e., ηₜₒₜₐₗ=mean collapsed coverage/estimated input by ddPCR copy number quantification).

At a decision step 1335, it is determined whether the conversion efficiency is within an acceptable range. If the conversion efficiency is not within an acceptable range, then method 1300 returns to step 1315. However, if the conversion efficiency is within an acceptable range, then method 1300 proceeds to a step 1340. In a step 1340, a diagnostic decision and/or treatment decision is made.

In some embodiments, the methods and systems described herein are configured to operate on and include a digital processing device. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

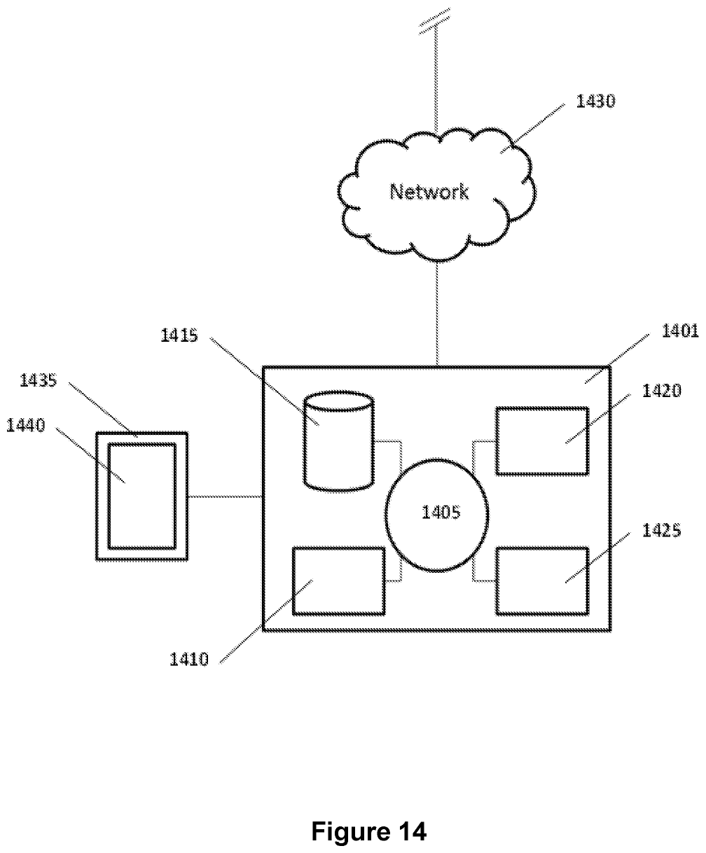

Referring to FIG. 14, in a particular embodiment, an exemplary digital processing device 1401 is programmed or otherwise configured to quantify low molecular weight nucleic acid molecules. The device 1401 can regulate various aspects of the quantitation method of the present disclosure, such as, for example, determining, from raw or normalized data, amounts of total nucleic acid targets or high molecular weight nucleic acid targets; and/or comparing and calculating total and high molecular weight nucleic acid target amounts to arrive at an amount of low molecular weight nucleic acid targets. In this embodiment, the digital processing device 1401 includes a central processing unit (CPU, also “processor” and “computer processor” herein) 1405, which can be a single core or multi core processor, or a plurality of processors for parallel processing. The digital processing device 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The digital processing device 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the device 1401, can implement a peer-to-peer network, which may enable devices coupled to the device 1401 to behave as a client or a server.

Continuing to refer to FIG. 14, the CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and write back. The CPU 1405 can be part of a circuit, such as an integrated circuit. One or more other components of the device 1401 can be included in the circuit. In some cases, the circuit is an application specific integrated circuit (ASIC) or a field programmable gate array (FPGA).

Continuing to refer to FIG. 14, the storage unit 1415 can store files, such as drivers, libraries and saved programs. The storage unit 1415 can store user data, e.g., user preferences and user programs. The digital processing device 1401 in some cases can include one or more additional data storage units that are external, such as located on a remote server that is in communication through an intranet or the Internet.

Continuing to refer to FIG. 14, the digital processing device 1401 can communicate with one or more remote computer systems through the network 1430. For instance, the device 101 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

### Non-Transitory Computer Readable Storage Medium

In some embodiments, the methods and systems disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB.NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon Kindle® Basic Web, Nokia® Browser, Opera Software® Opera® Mobile, and Sony® PSP™ browser.

### Software Modules

In some embodiments, the methods and systems disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the methods and systems disclosed herein include one or more databases, or use of the same. In view of the disclosure provided herein, those of skill in the art will recognize that many databases are suitable for storage and retrieval of nucleotide sequence information, quantitation information, target copy number or count number of either high or low nucleic acid targets or total nucleic acid targets. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

The following illustrative examples are representative of embodiments of the systems and methods described herein and are not meant to be limiting in any way.

### Example 1—Multi-Point ddPCR Copy Number Quantification (CNQ)

In an example of multi-point ddPCR copy number quantification, FIG. 2 shows a schematic plot 200 of ddPCR counts as a function of amplicon length (lₐ). ddPCR counts for each target (N_{c}) were plotted as a function of amplicon length and a linear regression line was fit through the data points. The linear fit was extended back to an amplicon length of 1 (or zero), and the value on the y-axis at that point represents the actual real count (or measured count (N_{targets})) of target fragments in the cfDNA subsample.

FIG. 3A shows a plot 300 of droplet fluorescence from an experiment in which high molecular weight gDNA fragments were selectively amplified in a cfDNA sample. FIG. 3B shows a representative plot 310 of the representative fragment size distribution of the sample used in plot 300 shown in FIG. 3A. Referring to FIG. 3A, plot 300 shows a cluster of droplets containing the target gDNA amplicon. The primer pair used in this amplification reaction selectively counted the higher molecular weight gDNA fragments in the cfDNA sample and did not amplify the lower molecular weight cfDNA fragments (average size about 160 bp). Referring to FIG. 3B, the unshaded area of plot 310 indicates the approximate range of higher molecular weight fragments that were amplified using the gDNA-specific primer pair.

FIG. 4 shows a plot 400 of correcting the linear fit count (real count) for large gDNA contamination. To correct for high molecular weight gDNA contamination in a cfDNA subsample, the gDNA count (N_{gDNA}) is subtracted from the linear fit count (N_{targets}) to generate a real corrected count (N_{corr.}) for copy number (i.e., N_{corr.}=N_{targets}−N_{gDNA}). The linear fit line is adjusted downward and the value on the y-axis at that point represents the actual corrected real count of target fragments in the cfDNA subsample.

### Example 2—Evaluation of ddPCR Copy Number Quantification

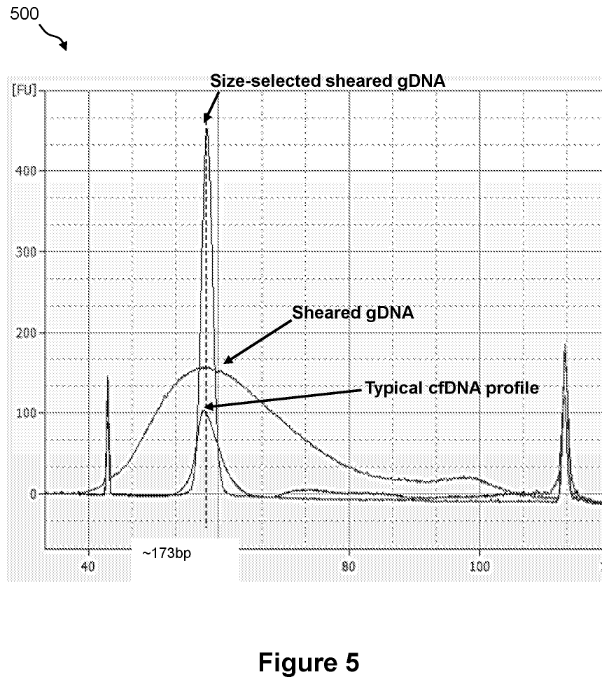

In an example of the evaluation of ddPCR copy number quantification, FIG. 5 shows a plot 500 of the fragment size distribution of size-selected genomic DNA used to evaluate ddPCR counts as a function of amplicon length.

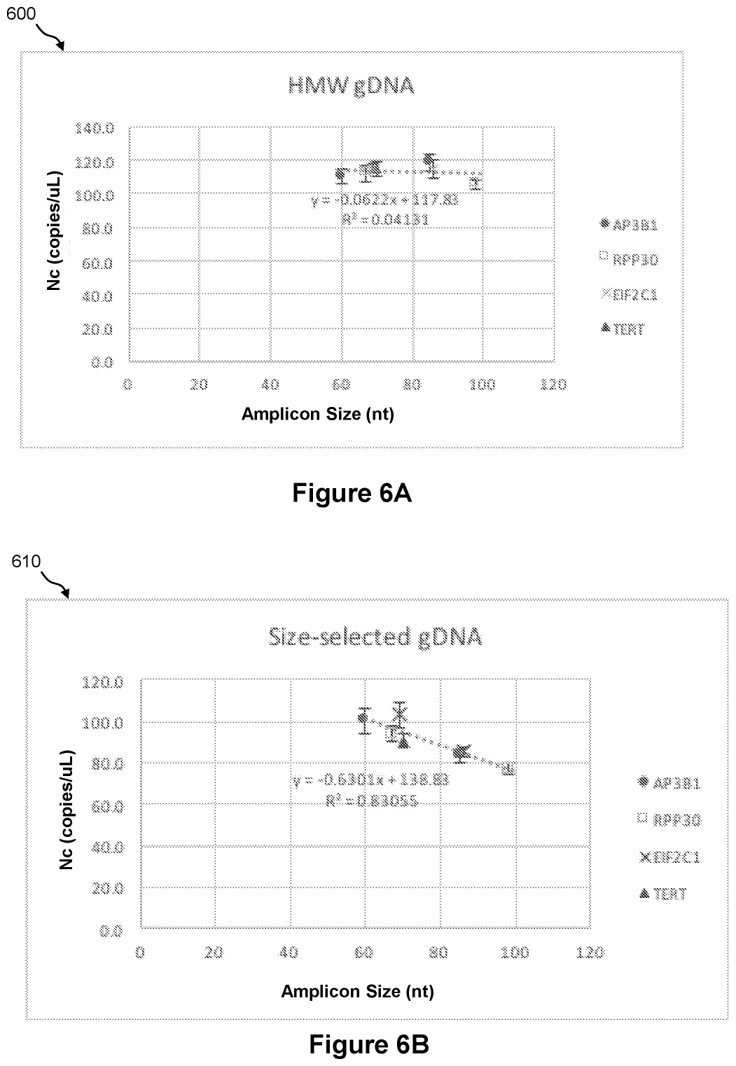

FIGS. 6A and 6B show a plot 600 of ddPCR counts (N_{c}) as a function of amplicon length in an un-sheared high molecular weight gDNA sample and a plot 610 of ddPCR counts (N_{c}) as a function of amplicon length in the size-selected sheared gDNA of FIG. 5, respectively. Referring to FIG. 6A, the data showed that in the unsheared high molecular weight gDNA sample, the number of ddPCR counts (N_{c}) across the different amplicon sizes was substantially the same. Referring to FIG. 6B, the data showed that in the size-selected sheared gDNA sample, a downward trend in the number of counts (N_{c}) was observed as amplicon length was increased; i.e., with increasing amplicon size, the number of counts was decreasing.

To evaluate the relationship between target amplicon length and number of counts, genomic DNA was sheared, size-selected for fragments of about 173 bp in size, and amplified using a set of primer pairs and probes selected to yield 7 different length amplicons (e.g., ranging from about 60 to about 100 nt) across 4 different target regions of the genome (i.e., AP3B1, RPP30, EIF2C1, and TERT). For each target amplicon, a ddPCR count was determined and data plotted as target copies/μL (i.e., Nc (cp/μL)).

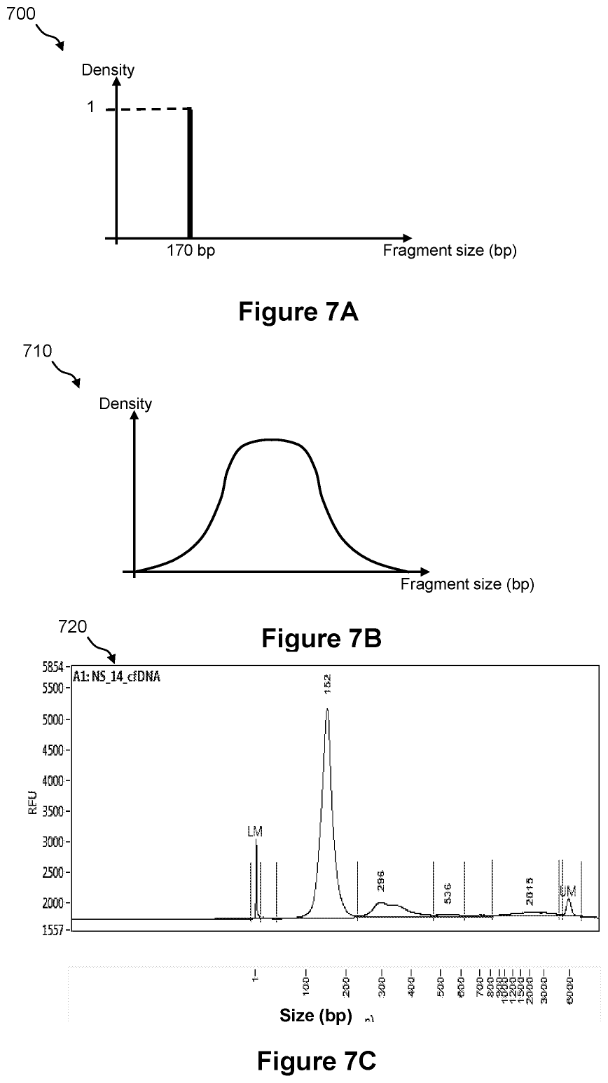

In a simplified example, wherein all the fragments in a sample are the same length (e.g., 170 bp), the probability of “amplicon capture” can be calculated. FIG. 7A shows a density plot 700 of fragment size distribution for a single size fragment. In this example, probability (P) of an amplicon of length lₐ on a fragment of length l_{ƒ} is a linear function of amplicon length and can be described as:

$$P ( {l}_{f} , {l}_{a} ) = \frac{{l}_{f} - {l}_{a} + 1}{{l}_{f}} = ( - \frac{1}{{l}_{f}} ) {l}_{a} + \frac{{l}_{f} + 1}{{l}_{f}}$$

This is because the first position of the left primer has a total of l_{ƒ} positions to land on. Of these, the last lₐ−1 position are not favorable since, then, the primer pair cannot fully land on the fragment due to the right primer being partially landing at best. As a result, only l_{ƒ}-(lₐ−1) of the l_{ƒ} positions allow for full landing, hence the l_{ƒ}−1ₐ+1/l_{ƒ} probability.

The number of counts N_{c} as a function of fragment length (l_{ƒ}) and amplicon length (lₐ) becomes the real number of fragments (N) multiplied by the probability P(l_{ƒ}, lₐ):

N_{c}(l_{ƒ},lₐ)=N×P(l_{ƒ},lₐ)∝lₐ

The simplified example can be extended to a distribution of a range of different fragment sizes. FIG. 7B shows a plot 710 of a schematic density histogram of the frequency of fragment density as a function of fragment size (bp) for a hypothetical sample with continuous fragment size distribution. The equation becomes a continuous integration over the fragment length distribution, with the lower bound of lₐ (as fragment of size smaller than lₐ cannot be captured):

$${N}_{c} ( {l}_{a} ) = N . {\int }_{{l}_{a}}^{{l}_{2}} {ρ}^{*} ( l ) . P ( l , {l}_{a} ) . d l$$

One can expand the equation as follows:

$$ρ ( l ) = \frac{i ( l )}{{\int }_{{l}_{1}}^{{l}_{2}} i ( l ) . d l} . dl \to {ρ}^{*} ( l ) = \frac{i ( l )}{K} ( K ≡ {\int }_{{l}_{1}}^{{l}_{2}} i ( l ) . d l )$$

$${N}_{c} ( {l}_{α} ) = N . {\int }_{{l}_{a}}^{{l}_{2}} {ρ}^{*} ( l ) . P ( l , {l}_{α} ) . dl = N . {\int }_{{l}_{a}}^{{l}_{2}} {ρ}^{*} ( l ) . \frac{l - {l}_{α} + 1}{l} . d l = - N . {\int }_{{l}_{a}}^{{l}_{2}} \frac{{ρ}^{*} ( l )}{l} . d l + N . ( 1 + {\int }_{{l}_{a}}^{{l}_{2}} \frac{{ρ}^{*} ( l )}{l} . d l )$$

$$α ≡ {{\int }^{{l}_{2}}}_{{l}_{a}} \frac{{ρ}^{*} ( l )}{l} . d l \to {N}_{c} ( {l}_{α} ) = - N α . {l}_{α} + N ( 1 + α ) ≃ - N α . {l}_{α} + N ( since α \text{∼} 𝒪 ( {10}^{- 3} ) )$$

For the type of distributions of fragments that are observed in a cfDNA sample, shown in FIG. 7C, with assumption that only a negligible portion of the fragments have a size smaller than 100 bp, then α becomes a constant, rendering N_{c} a linear function of N:

$${N}_{c} ( {l}_{a} ) = N ( 1 + {K}_{1} . ln ( {l}_{a} ) . {l}_{a} + {K}_{2} . ln ( {l}_{a} ) ) \\ α ≡ {\int }_{{l}_{a}}^{{l}_{2}} \frac{{ρ}^{*} ( l )}{l} . d l = {\int }_{{l}_{a}}^{{l}_{f = 100}} \frac{{ρ}^{*} ( l )}{l} . d l + {\int }_{{l}_{f} = 100}^{{l}_{2}} \frac{{ρ}^{*} ( l )}{l} . d l = c t e .$$

Even for a complicated fragment size distribution which has fragments of size <100 bp which is relatively constant, the equation can be expanded as follows:

$${N}_{c} ( {l}_{a} ) = - N . α ( {l}_{a} ) . {l}_{a} + N . ( 1 + α ( {l}_{a} ) ) = N ( 1 + α ( {l}_{a} ) . ( 1 - {l}_{a} ) )$$

$$α ( {l}_{a} ) = {\int }_{{l}_{a}}^{{l}_{2}} \frac{{ρ}^{*} ( l )}{l} . dl = {\int }_{60}^{{l}_{2}} \frac{{ρ}^{*} ( l )}{l} - {\int }_{{l}_{a}}^{60} \frac{{ρ}^{*} ( l )}{l} . dl == {K}_{1} - {K}_{2} ( ln ( {l}_{a} ) - ln 60 ) = {K}_{3} - {K}_{2} . ln ( {l}_{a} ) \\ \to {N}_{c} ( {l}_{a} ) = N ( {K}_{4} + {K}_{5} . ln ( {l}_{a} ) . {l}_{a} + {K}_{5} . ln ( {l}_{a} ) )$$

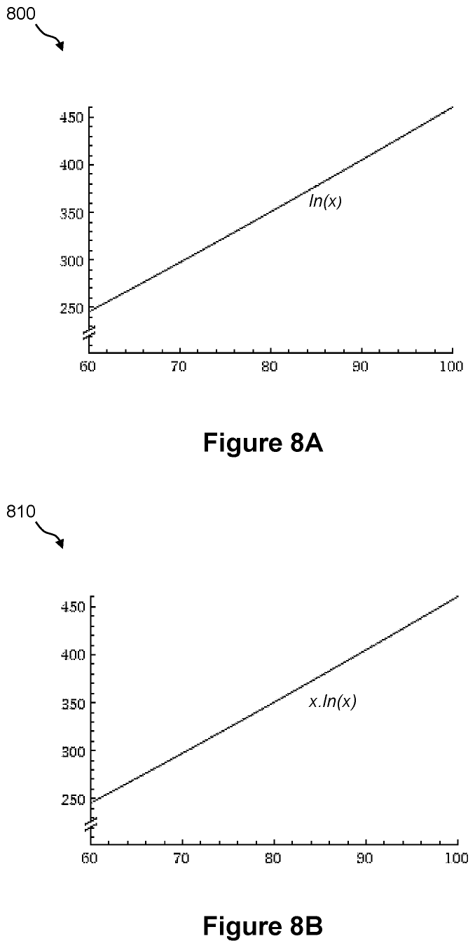

In this case, also, as shown in FIG. 8A and FIG. 8B, components of the function are linear functions of lₐ (plot 800 for functions ln(x) and plot 810 for the function x.ln(x)), and a linear combination of these functions means that the measured count becomes a linear function of the real counts.

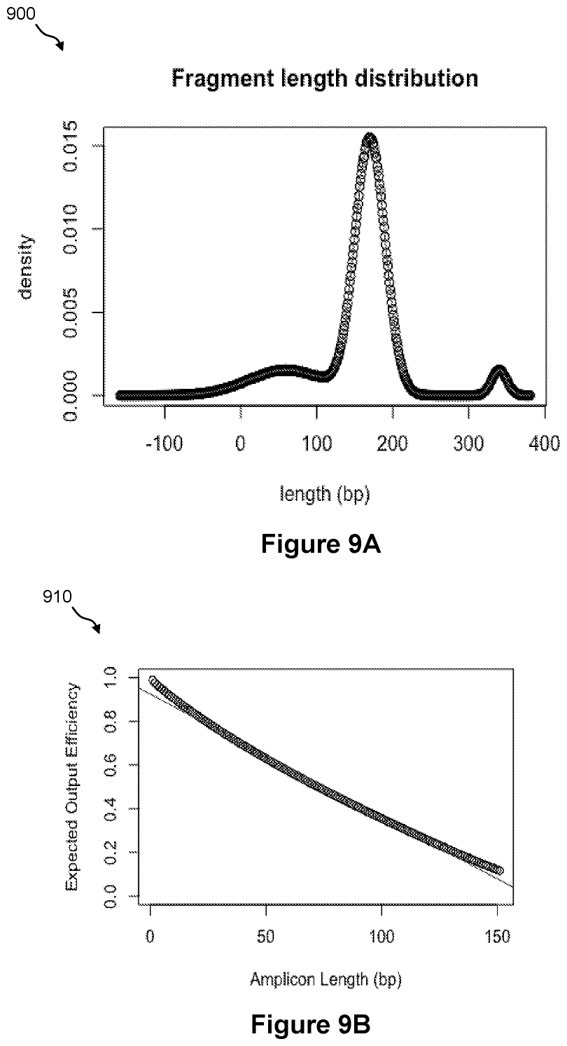

A simulation tool can be used to generate different hypothetical fragment length distributions for cfDNA. FIGS. 9A and 9B show a plot 900 of the simulation of fragment density as a function of fragment length and a plot 910 of the simulation of the expected output efficiency as function of amplicon length, respectively. The simulation showed that the linear behavior was consistent for a range of cfDNA fragment sizes.

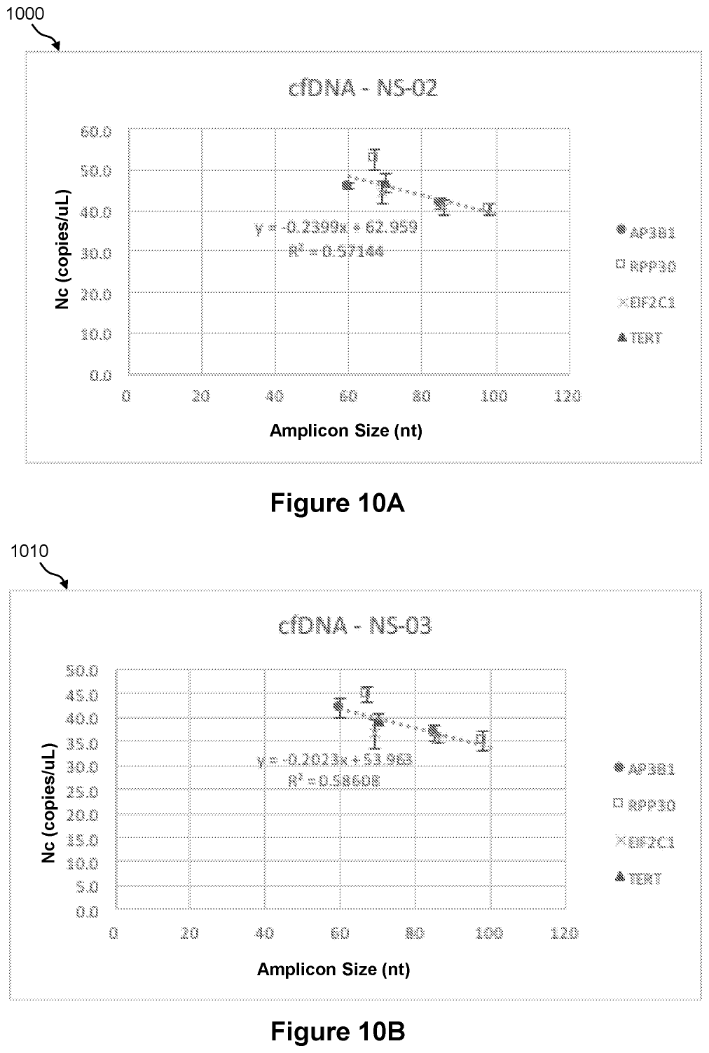

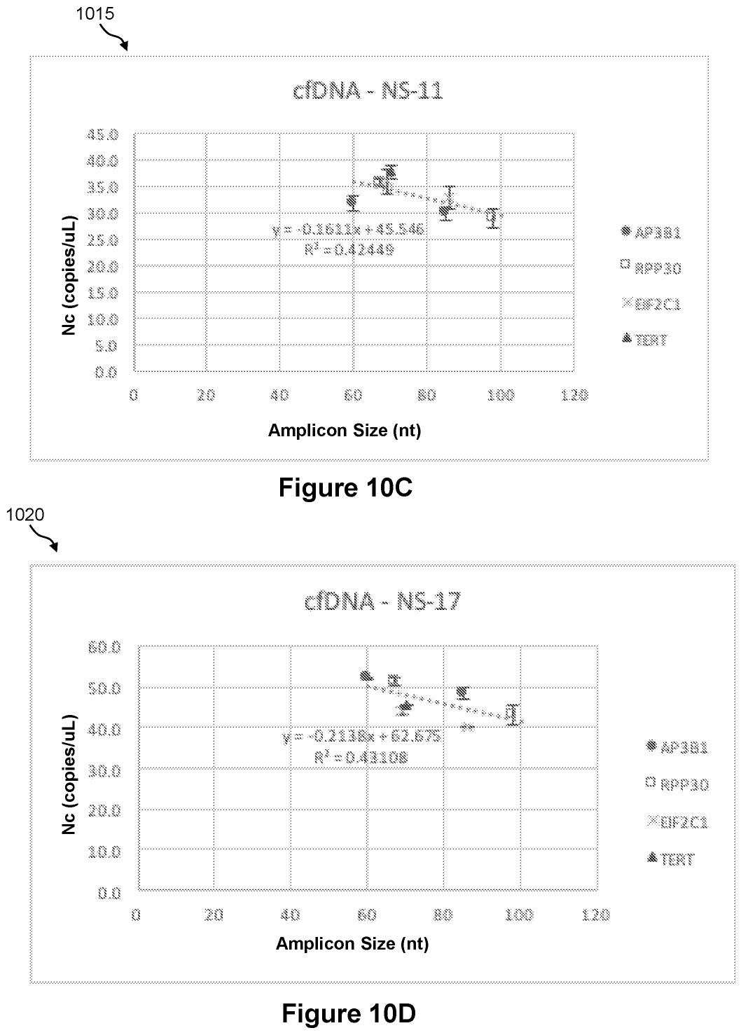

FIGS. 10A, 10B, 10C, and 10D show plots 1000, 1010, 1015, and 1020 of ddPCR counts (N_{c}) as a function of amplicon length for 4 different cfDNA samples, NS-02, NS-03, NS-11, and NS-17, respectively. In this experiment, a set of primer pairs and probes selected to yield 7 different length amplicons (e.g., ranging from about 60 to about 100 nt) across 4 different target regions of the genome (i.e., AP3B1, RPP30, EIF2C1, and TERT) were used. For each target amplicon, a ddPCR count was determined. The data showed that for all cfDNA samples, the expected downward trend in the number of counts (N_{c}) was observed as amplicon length was increased; i.e., with increasing amplicon size, the number of counts was decreasing.

The experiment was repeated 3-4 times (n=3-4) using cfDNA samples NS-2, NS-3, and NS-11. Table 1 below shows the measurement variation for each cfDNA sample. The data showed that ddPCR copy number quantification in cfDNA was consistent and repeatable.

TABLE 1
Measurement variation

N (cp/μL)*

| Sample | #1 | #2 | #3 | #4 | Avg N_{c} | SD |
|---|---|---|---|---|---|---|
| NS-2 | 63.53 | 62.29 | 63.77 | 62.24 | 62.95 | 0.8 |
| NS-3 | 58.3 | 51.8 | 53.3 | 52.2 | 53.9 | 3 |
| NS-11 | 58.3 | 54.4 | 54.1 | — | 55.6 | 2.34 |

*copies/μL

The day-to-day variation (day 1 vs. day 2) in ddPCR copy number quantification was evaluated using 4 cfDNA samples (n=2-3). Table 2 below shows the copy number count (cp/μL) for each cfDNA sample. The data showed that the ddPCR copy number count (N) was fairly consistent (within about 5 to 10%) between day 1 and day 2.

TABLE 2
Day-to-day variation

N (cp/μL)*

| Sample | Day 1 | Day 2 |
|---|---|---|
| NS-2 | 62.96 | 64.34 |
| NS-14 | 35.44 | 37.27 |
| NS-15 | 36.27 | 38.5 |
| NS-17 | 34.58 | 41.78 |

### Example 3—Estimation of Conversion Efficiency Based on ddPCR Copy Number Quantification

To evaluate method 1100 of FIG. 11, three cfDNA samples (NS_14, NS_15, and NS_17) were used in a cfDNA sequencing and analysis workflow that included an enrichment step using a non-small cell lung cancer (NSCLC) enrichment panel. Table 3 below shows the conversion efficiency for each cfDNA sample based on ddPCR copy number quantification. In this example, the calculated conversion efficiency was about 25%.

TABLE 3
Conversion efficiency based on CNQ

| | | Estimated | |
|---|---|---|---|
| | Mean | input by | CNQ |
| | collapsed | ddPCR CNQ | conversion |
| Sample | coverage | (hGE) | efficiency |
| NS_14 | 6,475 | 26,464 | 24.5% |
| NS_15 | 4,147 | 15,444 | 26.9% |
| NS_17 | 3,510 | 14,681 | 23.9% |

To evaluate how ddPCR copy number quantification of cfDNA compares to indirect quantification using a Fragment Analyzer™ (Advanced Analytical Technologies), the amount of DNA in 12 cfDNA samples was determined using both methods. In addition, quantification of size-selected gDNA was also performed using ddPCR copy number quantification and Fragment Analyzer™ quantification.

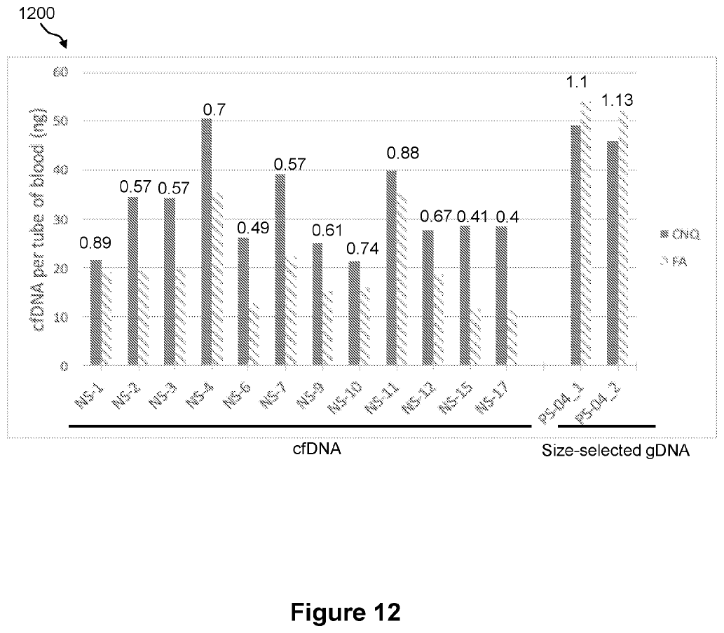

FIG. 12 shows a bar graph 1200 of a comparison of cfDNA quantification using ddPCR copy number quantification and Fragment Analyzer™ quantification. For all cfDNA samples, the amount of cfDNA per tube of blood (ng) measured using ddPCR CNQ was higher compared to the amount measured using the Fragment Analyzer™. The number above the set of bars for each cfDNA sample is the ratio of the Fragment Analyzer™ quantification/ddPCR copy number quantification. This graph suggested that Fragment Analyzer™ under-quantifies the amount of cfDNA sample. In contrast to the lower measurement of cfDNA using Fragment Analyzer™ quantification compared to ddPCR quantitation, Fragment Analyzer™ quantification of gDNA reported a higher amount of gDNA. This difference in quantification of cfDNA and gDNA using ddPCR copy number quantification and Fragment Analyzer™ quantification may be due to specific characteristics of cfDNA.

To compare estimation of conversion efficiency based on ddPCR copy number quantitation to estimation of conversion efficiency based on Fragment Analyzer™ (FA) quantification, a subsample of the cfDNA samples described with reference to Table 3 were quantified using a Fragment Analyzer™ and the calculated FA input (hGE) was used to determine the FA conversion efficiency. Table 4 below shows a comparison of the calculated conversion efficiency for ddPCR versus Fragment Analyzer™ (FA) quantification of cfDNA. In this example, the estimation of copy number equivalents (“FA input (hGE)”) based on Fragment Analyzer™ quantification of cfDNA was lower than the copy number estimation based on ddPCR copy number quantification (“CNQ input (hGE)”) and consequently, the calculated FA conversion efficiency was higher.

TABLE 4
Conversion efficiency for ddPCR CNQ vs. Fragment Analyzer ™

| | Mean | CNQ | CNQ | FA | FA |
|---|---|---|---|---|---|
| | collapsed | input | conversion | input | conversion |
| Sample | coverage | (hGE) | efficiency | (hGE) | efficiency |
| NS_14 | 6,475 | 26,464 | 24.5% | 20,378 | 31.8% |
| NS_15 | 4,147 | 15,444 | 26.9% | 6,178 | 67.1% |
| NS_17 | 3,510 | 14,681 | 23.9% | 6,019 | 58.3% |

The experiment was repeated to obtain a second set of measurements. Table 5 below shows the comparison of the calculated conversion efficiency for ddPCR copy number quantitation versus Fragment Analyzer™ (FA) quantification of cfDNA for the repeat experiment. In this example, the conversion efficiency calculated based on ddPCR quantification of unique molecule input was consistent with the conversion efficiency shown in Table 4. However, the conversion efficiency calculated based on Fragment Analyzer™ quantification of genome equivalent input was inconsistent.

TABLE 5
Conversion efficiency for ddPCR CNQ vs. Fragment
Analyzer ™ (repeat)

| | Mean | CNQ | CNQ | FA | FA |
|---|---|---|---|---|---|
| | collapsed | input | conversion | input | conversion |
| Sample | coverage | (hGE) | efficiency | (hGE) | efficiency |
| NS_14 | 6,475 | 26,053 | 24.9% | 24,750 | 26.2% |
| NS_15 | 4,147 | 13,981 | 29.7% | 13,702 | 30.3% |
| NS_17 | 3,510 | 11,864 | 29.6% | 12,339 | 28.4% |

While preferred embodiments of the present invention have been shown and described herein, it will be understood to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A method for quantifying low molecular weight nucleic acid molecules in a biological sample comprising said low molecular weight nucleic acid molecules and high molecular weight nucleic acid molecules, comprising:
a. on a first subsample of said biological sample, quantifying total nucleic acid targets, wherein said total nucleic acids comprise both low molecular weight nucleic acid targets and high molecular weight nucleic acid targets;
b. on a second subsample of said biological sample, quantifying one or more high molecular weight nucleic acid targets, wherein said high molecular weight nucleic acid targets are longer than said low molecular weight nucleic acid targets; and
c. quantifying said low molecular weight nucleic acid targets in said biological sample by comparing an amount of said high molecular weight nucleic acid targets and said low molecular weight nucleic acid targets.

2. The method of claim 1, wherein said high molecular weight nucleic acid targets, said low molecular weight nucleic acid targets, or both comprise DNA molecules.

3. The method of claim 1 or 2, wherein digital PCR (dPCR) is used to quantify one or more of said high molecular weight nucleic acid targets, one or more of said low molecular weight nucleic acid targets, or both of said high molecular weight nucleic acid targets and said low molecular weight nucleic acid targets.

4. The method of any one of claims 1 to 3, wherein said low molecular weight nucleic acid targets are shorter than about 700 base pairs.

5. The method of any one of claims 1 to 4, wherein said low molecular weight nucleic acid targets are between about 150 to about 190 base pairs.

6. The method of any one of claims 1 to 5, wherein said high molecular weight nucleic acid targets are longer than about 700 base pairs.

7. The method of any one of claims 1 to 6, wherein said high molecular weight nucleic acid targets are between about 700 to about 2000 base pairs.

8. The method of any one of claims 1 to 7, wherein said low molecular weight DNA targets comprise cell-free DNA (cfDNA) present when said biological sample was obtained from an individual.

9. The method of any one of claims 1 to 8, wherein said high molecular weight DNA targets comprise genomic DNA inside a cell when said biological sample was obtained from an individual.

10. The method of any one of claims 1 to 9, wherein said low molecular weight nucleic acid targets are highly conserved regions of the genome.

11. The method of any one of claims 1 to 10, wherein said high molecular weight nucleic acid targets are highly conserved regions of the genome.

12. The method of any one of claims 1 to 11, wherein the average length of said low molecular weight nucleic acid targets is less than about 300 base pairs.

13. The method of any one of claims 1 to 12, wherein the average length of said low molecular weight nucleic acid targets is less than about 170 base pairs.

14. The method of any one of claims 1 to 13, wherein the average length of said high molecular weight nucleic acid targets is greater than about 300 base pairs.

15. The method of any one of claims 1 to 14, wherein the average length of said high molecular weight nucleic acid targets is greater than about 700 base pairs.

16. The method of any one of claims 1 to 15, wherein said low molecular weight nucleic acid targets comprise a plurality of low molecular weight nucleic acid targets selected to yield amplicons of different lengths across a genome.

17. The method of any one of claims 1 to 16, wherein said high molecular weight nucleic acid targets comprise a plurality of high molecular weight nucleic acid targets are selected to yield amplicons of different lengths across a genome.

18. The method of any one of claims 1 to 17, wherein said low molecular weight nucleic acid targets, said high molecular weight nucleic acid targets, or both said low molecular weight nucleic acid targets and said high molecular weight nucleic acid targets are quantified by a plurality of primer pairs that selectively hybridize to highly conserved regions of the genome.

19. The method of any one of claims 1 to 18, wherein said plurality of primer pairs used to quantify said low molecular weight nucleic acid targets are selected to yield at least two or more different length amplicons across at least two or more different target regions of the genome.

20. The method of any one of claims 1 to 19, wherein said plurality of primer pairs used to quantify said low molecular weight nucleic acid targets are selected to yield at least 7 different length amplicons across at least 4 different target regions of the genome.

21. The method of any one of claims 1 to 20, wherein said low molecular weight nucleic acid targets, said high molecular weight nucleic acid targets, or both said low molecular weight nucleic acid targets and said high molecular weight nucleic acid targets are quantified by a plurality of primer pairs that selectively hybridize to highly conserved regions of the genome.

22. The method of any one of claims 1 to 21, wherein the plurality of primer pairs to quantify the low molecular weight nucleic acid targets are selected to yield at least two or more different length amplicons across at least two or more different target regions of the genome.

23. The method of any one of claims 1 to 22, wherein the biological sample is selected from the list consisting of whole-blood, plasma, serum, saliva, lymph, and urine.

24. A method for determining a conversion efficiency in one or more steps of a nucleic acid sequencing and analysis workflow, the method comprising:
a. performing a step of said nucleic acid sequencing and analysis workflow on a sample comprising low molecular weight nucleic acid targets and high molecular weight nucleic acid targets; and
b. quantifying, using a digital PCR (dPCR) amplification reaction, a number of said low molecular weight nucleic acid targets in said sample before and after said step of the sequencing and analysis workflow, and comparing the number of low molecular weight nucleic acid targets in the sample before and after said sequencing and analysis workflow to determine said conversion efficiency of the step.

25. The method of claim 24, wherein said dPCR amplification reaction comprises droplet digital polymerase chain (ddPCR).

26. The method of claim 24 or 25, wherein said one or more steps of said sequencing and analysis workflow is selected from the group consisting of: DNA isolation, enrichment, ligating adaptors, performing a universal amplification step, attaching barcodes, and sequencing.

27. The method of any one of claims 24 to 26, wherein said step of said sequencing and analysis workflow is a plurality of steps selected from the group consisting of: DNA isolation, enrichment, ligating adaptors, performing a universal amplification step, attaching barcodes, and sequencing.

28. The method of any one of claims 24 to 27, wherein said low molecular weight nucleic acid targets are quantified by a plurality of primer pairs that selectively hybridize to highly conserved regions of the genome.

29. The method of claim 24, wherein quantifying comprises determining a first target count using a first set of one or more primer pairs that amplify one or more first regions of the genome and a second target count using a second set of one or more primer pairs that amplify one or more second regions of the genome.

30. The method of any one of claims 24 to 28, wherein estimating the conversion efficiency comprises comparing the second target count and the first target count.

31. The method of any one of claims 24 to 29, wherein the step is repeated if said conversion efficiency is less than about 20%.

32. The method of any one of claims 24 to 31, wherein the average length of said low molecular weight nucleic acid targets are is less than about 300 base pairs.

33. The method of any one of claims 24 to 32, wherein the average length of said low molecular weight nucleic acid targets is less than about 170 base pairs.
